(19) United States
(12) Patent Application Publication
(10) Pub. No.: US 2020/0146836 A1
(43) Pub. Date: May 14, 2020

(54) IMPLANT DEVICE FOR PERFORMING POSTERIOR SPINAL ARTHRODESIS AT A FACET JOINT

(71) Applicant: SC MEDICA, Strasbourg (FR)

(72) Inventors: Robin SROUR, Strasbourg (FR); Camille SROUR, Paris (FR)

(21) Appl. No.: 16/631,602

(22) PCT Filed: Jul. 19, 2018
(86) PCT No.: PCT/EP2018/069690

§ 371 Date: Jan. 16, 2020

(30) Foreign Application Priority Data
Jul. 17, 2017 (FR) 1756759

(51) Int. Cl. A61F 2/44; A61B 17/70

(52) U.S. Cl. CPC A61F 2/4405; A61F 2002/2835; A61F 2/447; A61B 17/7064

(57) ABSTRACT

An implant device for performing posterior spinal arthrodesis at a facet joint, includes a flat element, of constant thickness, having, in order to receive a bone graft, a large central opening so as to form a substantially annular shape. The device is intended to be inserted into a cavity previously produced at the location of the facet joint that is to be immobilised. The element is D-shaped, the rounded side constituting the distal edge for insertion into the cavity, while the proximal opposing edge, which is straight, includes a device for securing to a holding and positioning tool. The opposing faces of the flat element have raised areas that include notches arranged perpendicular to the direction of insertion, each of the notches having a tooth profile with two sides.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to an implant for performing a posterior spinal arthrodesis at a facet joint.

### 2. Description of Related Art Including Information Disclosed Under 37 CFR 1.97 and 37 CFR 1.98.

Such a procedure, done by posterior route, makes it possible to immobilize two vertebrae by using each of the lower articular apophyses of a vertebra with the opposite upper articular apophysis, of the adjacent lower vertebra.

Different techniques exist for performing a spinal arthrodesis. Through the posterior route, which is easiest, it is possible to use a plate or osteosynthesis bar(s) fastened using pedicle screws, or intervertebral cages of the type described for example in documents US 2011/230965, FR 2,946,245 and WO 02/03895, suitable for being placed in the intervertebral space after removal of the vertebral disc.

Posterior spinal arthrodesis at a facet joint has advantages relative to the other techniques frequently used. This technique is in particular less traumatic and has fewer risks.

This technique, however, requires using different means from those of other operating techniques, thus the characteristics of the intervertebral cages, such as those of the aforementioned documents, are not suitable for use for this operating mode.

Implant devices are already known that are suitable for being inserted in a facet joint, and in particular that described in document WO 2012154653, which consists of a corner-shaped element, the two opposite faces of which, each suitable for coming into contact with a facet, have a surface provided with reliefs to ensure the anchoring thereof, and which is pierced with multiple holes suitable for the development of the bone material after a bone graft is deposited, while the fastening is done using two screws, each screwed into an apophysis. This implant device remains complex, or more particularly its implementation is complex.

### BRIEF SUMMARY OF THE INVENTION

The present invention aims to propose an implant for performing a posterior spinal arthrodesis at a facet joint, which has a simpler design than the known devices, and which is also easier to implement.

The implant device for performing a posterior spinal arthrodesis at a facet joint according to the invention is characterized in that it assumes the form of a flat element of constant thickness, comprising, to receive a bone graft, a wide central opening imparting a substantially annular shape, and suitable for being introduced into a cavity made beforehand in the location of the facet joints to be immobilized; in that said element is T-shaped, the rounded side making up the distal edge for insertion into said cavity, while the opposite, rectilinear proximal edge includes means for securing to a handling and placement tool; and in that the opposite faces of said flat element include reliefs that consist of notches arranged perpendicular to the insertion direction, each of the notches having a toothed profile with two faces, one of which, that oriented on the side of said proximal edge, is perpendicular or substantially perpendicular to the main plane of said element, while the other face is inclined.

According to one additional feature of the implant device according to the invention, the flat element has, on the side of its rounded distal edge, a thickness that decreases, so as to impart a corner profile to this distal edge.

According to another additional feature of the device according to the invention, the means for securing to a handling and placement tool consists of a tapped hole, with axis parallel to the insertion direction.

The advantages and features of the implant device according to the invention will emerge more clearly from the following description relative to the appended drawing, which shows one non-limiting embodiment thereof.

### BRIEF DESCRIPTION OF THE SEVERAL VIEWS OF THE DRAWINGS

In the appended drawings:

FIG. 1 shows a planar view of the implant device according to the invention.

FIG. 2 shows a sectional view along axis AA′ of FIG. 1.

FIG. 3 shows a schematic view of the same implant in position after implantation, the right side between vertebrae L5 S1, and along the insertion direction.

FIG. 4 shows a schematic view of the same implant in the same position, but shown transparently and along a perpendicular plane.

### DETAILED DESCRIPTION OF THE INVENTION

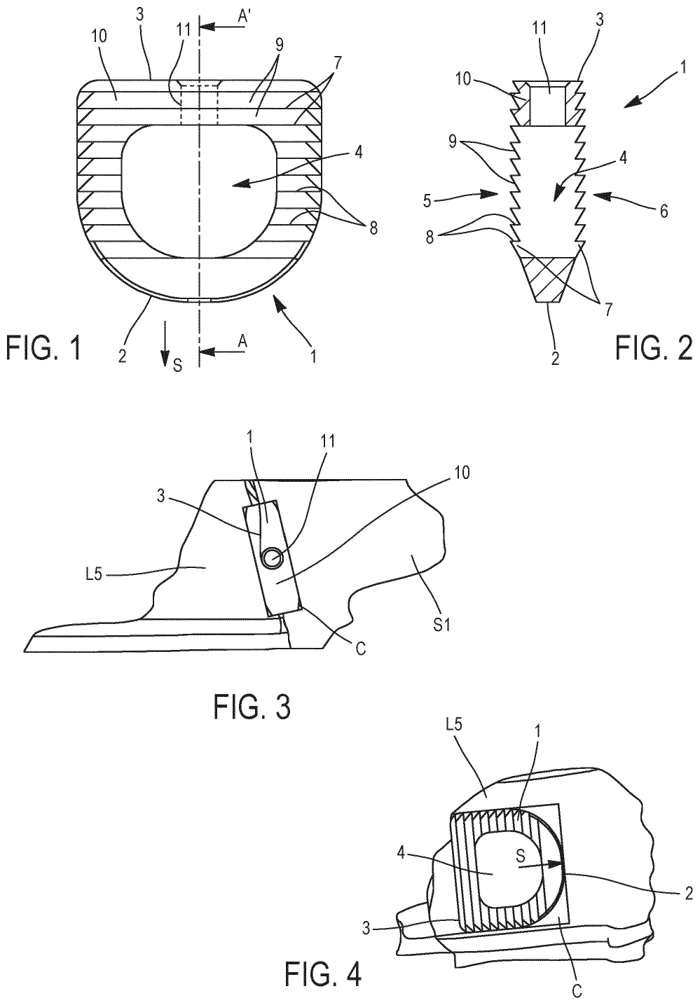

In reference to FIGS. 1 and 2, an implant device 1 is shown for performing an arthrodesis at a facet joint according to the invention.

It will be noted beforehand that the implant device 1 according to the invention is made, non-limitingly, from titanium.

This implant device 1 is substantially flat, as shown more specifically in FIG. 2, and assumes the shape of a D. It thus has a rounded distal side 2 and a straight proximal side 3, whereas centrally, a wide space 4 is freed.

Each of the two faces 5 and 6 of the implant device 1 is trimmed with notches 7 aligned transversely relative to the axis AA′, suitable for performing anchoring in the bone material. As can be seen in FIG. 2, each of the notches 7 comprises two faces, a face 8 arranged on the straight proximal side 3 and which is perpendicular or substantially perpendicular to the main plane of the implant device 1, and a face 9 arranged on the distal side 2, and which is inclined.

In this same FIG. 2, one can also see that, on the distal side 2, the two faces 5 and 6 come closer together, so as to give this distal side 2 a corner profile.

One can also see in these figures that the part 10 of the implant device 1, forming the proximal side 3, is pierced, along the axis AA′, with a hole 11, tapped to allow it to be subjected to the end of a placement tool.

In reference now to FIGS. 3 and 4, one can see an implant device 1 according to the invention placed in the location of the facet joint between vertebrae L5 and S1, the right side of which is partially shown, knowing that the left side, not shown, is equipped in the same way.

Before the placement, the bone material of the two vertebrae is cut out, at the facet joint, so as to create a cavity C, shared between the two vertebrae, in this case L5 and S1, and extending in the contact plane.

Then, the implant device 1 is introduced into the cavity C, after having deposited a bone graft in its space 4.

The introduction of the implant device 1 is done along the direction of the arrow S shown in FIG. 4, that is to say, parallel to the axis AA′ of FIG. 1 and by engaging the corner-shaped distal side 2 first.

The implant device 1 according to the invention is very simple to produce and implement.

1. An implant device for performing a posterior spinal arthrodesis at a facet joint, comprising:
a flat element of constant thickness, being comprised of a central opening so as to receive a bone graft, imparting a substantially annular shape, and suitable for being introduced into a cavity made beforehand in the location of the facet joints to be immobilized,
wherein said element is D-shaped, the rounded side making up the distal edge for insertion into said cavity, while the opposite, rectilinear proximal edge is comprised of means for securing to a handling and placement tool, and
wherein the opposite faces of said flat element include reliefs are comprised of notches arranged perpendicular to the insertion direction, each of the notches having a toothed profile with two faces, one of which, that oriented on the side of said proximal edge, is perpendicular or substantially perpendicular to the main plane of said element, while the other face is inclined.

2. The implant device according to claim 1, wherein the flat element has, on the side of its rounded distal edge, a thickness that decreases, so as to impart a corner profile to this distal edge.

3. The implant device according to claim 1, wherein the means for securing to a handling and placement tool consists of a tapped hole, with axis parallel to the insertion direction.
